(19) United States
(12) Patent Application Publication
(10) Pub. No.: US 2008/0149100 A1
(43) Pub. Date: Jun. 26, 2008

(54) Antiviral Heat Treatment

(76) Inventors: Gerrit Jan Van Holst, Enkhuizen (NL); Petrus Theodorus De Haan, Oestgeest (NL)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/613,708

(22) Filed: Dec. 20, 2006

(51) Int. Cl. A61M 16/16; A61F 7/12

(52) U.S. Cl. 128/204.17; 607/105; 607/113

(57) ABSTRACT

A method for treating viral infections in vivo in subjects in need of such treatment by applying heat to affected body tissue, use of an apparatus to provide heated moist gas or heated liquids to affected body tissue that is infected with a virus and apparatuses capable of providing heat to a body part that is infected with a virus.

The invention relates to the field of medicine. More specifically, it relates to a method for treating viral infections that comprises applying heat to an organ or body part comprising a virus load, apparatuses capable of applying heat to said organ or body part, and uses therefor. In particular, the invention is applicable to the combating of or treatment of viral infections of organs such as the skin and internal organs and body parts such as those of the respiratory system (lungs), the female genital tract (eg cervix), body extremities such as the hands, feet, and male and female sexual organs such as the penis, scrotal sac, vulva and pubic areas.

Viruses are obligate intracellular parasites and require the biochemical machinery of the host cell to replicate and spread. The genetic material of the virus is typically enclosed in a protein capsid, which in some cases may be surrounded by a lipid envelope.

Viruses are extremely diverse in terms of their structure and genetic complexities; some have RNA genomes encoding only a few genes while others have DNA genomes encoding up to 200 genes. Viruses bind to host cells via specific receptors. Following entry into the host cell, the virus uncoats, nucleic acid is released and is expressed to yield viral proteins. The viral genome is replicated and new progeny virus particles (virions) are assembled and released and these go on to infect neighbouring cells. Viral entry to cells is commonly made at mucosal sites. Replication typically occurs at epithelial surfaces, although this may happen in the case of certain virus infections by viraemia, that is to say, the virus particles get into the systemic circulation and are spread through it to infect other tissues.

Eukaryotes have developed a number of anti-microbial defence mechanisms based on the recognition of conserved molecular patterns that are shared by large groups of micro-organisms, including viruses. Double-stranded RNA (dsRNA) represents one such molecular pattern. In mammalian cells, a defence response that is induced by dsRNA is the induction of interferons, a class of proteins which can reduce viral spread by inhibiting viral gene expression and causing apoptosis of the infected cells. Another antiviral response that is induced by dsRNA is called RNA interference (RNAi) in animals and post-transcriptional gene silencing (PTGS) or RNA silencing in plants (Fire et al. Nature 1998, 391, 806-811; Ding, Curr. Opin. Biotechnology 2000, 11, 152-156; Hutvánger and Zamore Curr. Opin. Gen. Dev 2002, 12, 225-232). The key characteristic of RNAi is its remarkable sequence specificity: the infected organism responds to dsRNA by selectively degrading mRNAs that are homologous in sequence to the dsRNA inducer. Therefore, RNAi can be used to specifically block the expression of not only viral, but also host cell genes upon introduction of a homologous dsRNA. The central initiator molecules in RNA silencing are (long) dsRNA molecules, which are degraded by a dsRNA-targeted nuclease, denoted DICER to 21-23 nt fragments. These short interfering RNAs (siRNAs) subsequently target homologous cognate RNAs for degradation mediated by the RNA-initiated silencing complex (RISC), resulting in a low amount of cytoplasmic target RNA. If the target RNA is either a viral genome, antigenome or mRNA, RNAi-induced RNA degradation can specifically inhibit, or even entirely block, viral replication. Conversely, if the target RNA is of cellular origin, then RNAi can post-translationally block the expression of the cognate gene.

To overcome host RNA silencing, it has been found that a number of plant viruses encode RNA silencing suppressors (RSS). Published patent applications having international publication numbers WO 98/44097, WO 01/38512 and WO 02/057467 describe plant virus-derived RNA silencing suppressors and their use in plants. WO 02/057301 describes a plant virus-derived RSS and its use in animal cells, whereas Li et al., Science 296: 1319-1321, 2002, describes an insect virus-derived RSS and its activity in plant and insect cells. The use of vertebrate virus-derived RSS and their use in animal cells was described in international publication WO 04/035796. A study by Li et al. (Proc. Natl. Acad. Sci. USA 2004, 101, 1350-1355) showed that NS1 and E3L—proteins encoded by the mammalian viruses influenza and vaccinia, respectively—were able to suppress antiviral RNA silencing in Drosophilae cells, which strongly supports a role for RNAi as a natural antiviral response not only in plant cells but also in other non-plant eukaryote cells.

A further host defence against viruses is fever. It is commonly thought that fever takes advantage of the fact that many invading organisms tolerate a narrower temperature range than body tissues and are therefore more susceptible to increases in temperature, i.e. they may die from overheating before harm is done to human tissues. Fever also stimulates the immune system by increasing the production of antibodies and interferon.

Several studies have shown that viral replication in vitro can be suppressed by increases in temperature. For example, Sweet et al (Br. J. Exp. Path. 1978, 59, 373-380) described that Influenza A virus grown in organ cultures of ferret nasal turbinates was inactivated at pyrexial temperatures. The findings of Sweet et al. were supported by the results of studies in ferrets that were infected with the virus. In some animals, fever induced by the viral infection was suppressed by shaving the ferrets or by a treatment with an antipyretic drug. It was observed that mean viral titres in nasal washes decreased less rapidly in animals with a reduced temperature compared to control animals which did develop a fever (Husseini et al. (1982) J. of Infectious Diseases Vol. 145, 520-524). Thus, a correlation was observed between body temperature and viral titres of Influenza A virus. Conti et al. (Antimicr. Agents and Chemoth. 1999, Vol. 43, 822-829) studied the effect of brief (20 min. 45° C.) hyperthermic treatment (HT) on the replication of human rhinovirus in HeLa cells. It was observed that HT suppressed viral replication in vitro by more than 90% when applied at specific stages of the virus replication cycle.

Replication of viruses that cause the common cold, including rhinoviruses and coxsackieviruses, mainly occurs in the epithelial cells that line the upper respiratory tract. Steam inhalation is a well known home remedy for infections of the upper respiratory tract. This typically involves holding the head under a towel over a bowl of very hot water for a time period typically ranging from 5 to 20 minutes. A nasal decongestant or other aromatic compound may be added to the water. Aromatic oils mildly irritate the mucous membranes of the respiratory tract, increasing secretion of mucous and saliva, which can relieve sore throat and nasal congestion. An apparatus for these applications is known under the name of rhinotherm™. The invention described in the published patent application, publication number WO 03/97143 discloses an alternative apparatus to relieve common colds which blows a negatively ionised current of heated air into the nasal cavity.

Whereas steam inhalation is commonly used to relieve some of the symptoms of common colds, the evidence that steam inhalation has any beneficial effect on the course of the illness is very limited. One review on the use of heated humidified air (involving one or more treatments of 20-30 min) for the common cold concluded that although there was evidence that symptoms were relieved, there was no objective improvement in outcome measures such as viral shedding or viral titres in nasal washings (Singh M. Heated, humidified air for the common cold (Cochrane Review). In: The Cochrane Library, Issue 2, 2004). Thus, whereas an inhibitory effect of hyperthermia on viral replication in vitro has been convincingly shown, the therapeutic value of heat to combat viral infections in vivo has thus far remained elusive.

The present inventors have surprisingly observed that at increased temperatures, that is to say temperatures above that of normal body temperature, the RNAi activity in a population of animal host cells is enhanced compared to that in a similar population of animal host cells at normal body temperature. Based on this knowledge, the present inventors surmised that a heat treatment of a virus-infected organ or body part should have an inhibitory effect on virus replication in vivo, that may be sufficient to combat or treat the virus infection. A sufficient level of inhibition of virus replication requires heating to at least 2° C. above the normal physiological temperature of the organ or body part for a period sufficient to induce protein synthesis from heat induced genes. These conditions lead to a prolonged increase of RNAi in the tissue, which implies that the capacity to silence genes via RNAi remains elevated for up to about 96 hours or more, and generally for between about 6 hours up to about 96 hours. Typically, the higher the temperature, the shorter the time interval that is required for heating, subject to the proviso that the higher temperature is not so high as to be deleterious to the viability of tissue exposed to the heating temperature (compared to the pre-treatment situation).

According to the present invention there is provided a method for treating a viral disease in a mammal in need of such treatment, which comprises heating cells of virus-infected tissue of the said mammal with a heating apparatus to a heating temperature of at least 2° C. higher than that of the normal physiological body temperature for the said tissue. In a preferment the method is carried out at a temperature of from 2° C. to about 13° C. above that of the normal temperature of the tissue being treated. The types of tissue that the method of the invention may be applied to include virus-infected tissue that comprises cells that harbour virus particles from organs and body parts such as epithelial cells of the lungs, nasal passages, trachea, alveoli, and the like, and body parts such as the feet, hands, skin, sexual organs and parts thereof such as the penis and scrotal sac, vulva and other sexual organ architecture as appropriate. Also included within the ambit of virus-infected tissue are cells from organs such as the kidneys, liver, heart, and uterus, including parts thereof such as the cervix, clitoris, vulva (both labia major and labia minor), perineum and surrounding infected skin.

According to the present invention there is provided a method for inhibiting the replication of a virus in virus-infected cells of a tissue of a subject mammal in vivo that comprises heating said virus-infected tissue to a temperature of at least 2° C. but not more than 13° C., preferably from at least 2° C. to about 5° C. above the normal physiological temperature of said tissue for a duration of at least 1 hour, preferably 1-4 hours, more preferably 1-2 hours, most preferably 1-1.5 hours. In a further preferment of the method of the invention there is provided a method for inhibiting the replication of viruses in vivo in a virus-infected tissue of a subject mammal, comprising heating said tissue to a temperature of at least 6° C. but not more than 9° C. above its normal physiological temperature for up to 45 minutes such that apoptosis in the tissue is not induced, preferably from about 10 minutes to about 45 minutes. In a still further preferment of the method of the invention the heating temperature employed can be from at least 10° C. but not more than 13° C. above the normal physiological temperature of the tissue being treated for a duration of less than 10 minutes such that apoptosis is not substantially induced in the heated tissue. It is thought that this time interval is sufficient to induce protein synthesis from heat induced genes.

Virus infections occur more frequently in the respiratory tract than in any other organ. This might be expected when one considers the heavy and constant direct contact that these tissues have with the physical environment and thus exposure to micro-organisms such as viruses. According to the concept of the present invention, it is thought that the magnitude of the antiviral RNAi response is correlated with increase in temperature: increased temperatures enhance RNAi, whereas RNAi is relatively suppressed at lower temperatures. The temperature in cells of the respiratory tract is usually lower than that of other body parts, except for the temperature of specialised body parts such as the skin and testes. Breathing also results in local cooling of the respiratory tract (upper and lower respiratory tract) to a temperature below core body temperature. It is thought that this is why the respiratory tract appears more sensitive to viral infections than other body parts. Because of the lower cell temperatures in cells of the upper respiratory tract and skin, the antiviral RNAi response is reduced compared to that of cells of other body parts, so that the chance that a virus infection becomes established is increased.

In a preferred embodiment of the invention, the mammalian subject being treated is a human being suffering from a viral infection. As the normal physiological (core) temperature of a human being is approximately 37° C., the heat treatment of the invention typically comprises heating the tissue to a temperature of at least 39° C. Higher temperatures are however preferred since these will inhibit viral replication more efficiently. On the other hand, too high temperatures of the human tissues (e.g. over 42° C.) for too long such as over about 1 hour may prevent the enhancement of RNAi or may even induce cell death. The induction of heat shock proteins by (in vivo) heat treatment is well known in the art. Induction of a heat shock protein (HSP) can thus be taken as an indicator for a heat treatment according to the invention that suitably enhances RNAi without causing cell death. Thus, too harsh heat treatments which do not induce heat shock proteins (for example HSP70) are to be avoided in the application of this invention. For example, in a heating protocol of the invention a human tissue may be heated to a temperature lying within the range 37-42° C., preferably to 39-42° C., for a time interval of at least one hour, or in a different heating protocol of the invention a human tissue may be heated to a temperature lying within the range of from 43-46° C., for a time interval of not more than 1 hour, preferably not more than for from about 15-45 minutes. The temperature of the tissue to be treated with a method of the instant invention is typically lower than that of the temperature of the heated gas or heated liquid providing heat to the tissue.

The normal physiological temperature of a tissue to be treated may deviate significantly from normal physiological body temperature of a subject. In these cases, the heating temperature may be adjusted accordingly. For example, the temperature of the nasal passage in humans is 33° C. Furthermore, a study on thermal mapping of the airways in humans showed that during quiet breathing of room air the average temperature in the respiratory tract ranges from 32° C. in the upper trachea to 35.5° C. in the subsegmental bronchi (McFadden et al. J. Appl. Physiol. 1985;58(2):564-70). Thus, heating these tissues in a method of the present invention to temperatures that are higher than the normal physiological temperatures for such tissues, e.g. to 37° C. or 38° C. in the case of a human subject, may therefore be sufficient to achieve inhibition of viral replication in a tissue with a normal temperature below that of core body temperature.

The method of the invention to increase the antiviral response of the host cell by enhancing the degradation of viral nucleic acids via the RNAi machinery of the host cell, is not limited to any specific type of virus. In fact, any virus that is susceptible to RNAi-mediated degradation of its mRNA or genome or a part thereof, will be sensitive to heat-induced or heat-enhanced RNAi according to the invention.

In one embodiment, a method as described herein is used to inhibit replication of a virus that can enter mucosal sites and/or replicate in epithelial cells. Examples of such viruses include respiratory viruses and herpes viruses. Respiratory viruses are those causing infections of the upper and/or lower respiratory tract, including interstitial pneumonia. Upper respiratory tract infections include the common cold (rhinitis), influenza, laryngitis (inflammation of the voice box), pharyngitis (sore throat), sinusitis, tonsillitis, measles and croup (in children). There are over 200 viruses that can cause upper respiratory tract infections. These types of viruses are extremely contagious and are spread by direct contact, such as shaking hands, sharing food or drink, and kissing. They can also be spread through coughing and sneezing. A virus can be spread from the hands to the upper respiratory tract by touching the eyes, nose, or mouth.

Respiratory viruses include among others, Orthomyxoviridae such as Influenza-virus. Influenza-virus is an RNA virus which may spread through the population at given times of the year e.g. winter. Influenza has the highest morbidity and mortality, particularly of older adults, of all viral respiratory infections. There is commonly spontaneous recovery and resolution. Paramyxoviridae such as Parainfluenzavirus and Respiratory Syncytial Virus (RSV). Both are RNA viruses, that are significant causes of respiratory infection in infants and young children. The principle lesion is bronchiolitis, sometimes necrotizing, and less frequently, interstitial pneumonia.

Ebola virus belongs to the Filoviridae and can be transmitted by aerosols. It has been shown that aerogenic infected animals show cell-associated Ebola virus antigens present in airway epithelium, alveolar pneumocytes, and macrophages in the lung and pulmonary lymph nodes; extracellular antigen was present on mucosal surfaces of the nose, oropharynx and airways.

Coronaviruses are plus strand RNA viruses and include human Meta-Pneumo Virus, which can cause respiratory infections particularly in infants, and Severe Acute Respiratory Syndrome virus. The primary route of transmission for coronaviruses is respiratory or through fomites.

The Picornaviridae consist of the genus Enterovirus, Rhinovirus and Hepatovirus. Picornaviruses are transmitted by fecal and aerosol routes and are the causal agents of poliomyelitis (poliovirus), fever and hand, foot and mouth disease (coxsackieviruses), diarrhoea (enteroviruses), common cold (rhinoviruses) or hepatitis (hepatoviruses).

The Herpes virus family consists of more than 80 known viruses, divided into three classes; alpha, beta and gamma herpesviruses. The most common in humans are the alpha herpesviruses, which are usually fast replicating and are represented by Herpes Simplex virus type-1 (HSV-1) and type-2 (HSV-2), and Varicella Zoster Virus (VZV). Either HSV-1 and HSV-2 can infect the mouth (oral herpes, lip blisters, mouth blisters) or genitals (genital herpes). Usually, HSV-1 occurs in the oral region, and HSV-2 occurs in the genital region. HSV-1 usually causes herpes labialis commonly known as oral herpes, cold sores or fever blisters. These are highly infectious open sores that crust over before healing. HSV-2 primarily causes genital herpes in men and women. The most common signs and symptoms of genital herpes include recurrent clusters of blisters, bumps and rashes in genital areas. HSV-2 can also cause herpes labialis, although less often than HSV-1.

VZV or human herpesvirus type-3 can cause causes two diseases, namely chickenpox (varicella) and shingles (herpes zoster). After primary infection, VZV lies dormant in the roots of sensory nerves, in the spinal cord. When the infection is reactivated, it causes pain and a rash in the area supplied by the affected sensory nerves. These areas are known as dermatomes. The slowly replicating beta herpesviruses are represented by Cytomegalovirus (CMV) and Human Herpesvirus-6 and 7. CMV primarily infects epithelial and endothelial cells. It causes sub-clinical infection in the lung of immuno-competent hosts, and clinical infection in immuno-compromised patients. Infection of the lung can result in focal or diffuse interstitial pneumonia with or without hyaline membrane formation, intra-alveolar exudates, haemorrhage, and fibrosis.

The Human papillomaviruses (HPVs) are members of the Papillomavirus genus of the Papovaviridae family. The HPV genome consists of double-stranded circular DNA contained within an icosahedral capsid. More than 70 types of HPV are recognised. HPV infections of the genital tract can cause the development of cervical cancer, and when transmitted to the respiratory tract of a newborn child, juvenile-onset recurrent respiratory papillomatosis can result. HPV infections also cause warts on skin and in the genital, digestive and respiratory tract.

Examples of viruses whose replication is advantageously suppressed using a method of the invention include orthomyxovirus, influenza virus, paramyxovirus, parainfluenza virus, respiratory syncytial virus (RSV), filovirus such as Ebola virus, coronavirus such as hMPV and SARS virus, picornavirus such as rhinovirus, Coxsackie A and B virus, herpesvirus such as HSV-1, HSV-2, VZV, CMV and HPV.

In a preferred embodiment, especially in those cases where the tissue resides in the respiratory system, said tissue is heated by exposing the tissue to a heated and humidified gas, capable of heating the tissue. Thus, in a method of the invention the duration of the heat treatment is at least two times longer compared with known steam inhalation treatments, for example those included in the Cochrane study supra, wherein the subject typically receives a treatment for 20-30 min, optionally followed by additional treatments with 1-2 hours intervals. As described, known heat treatment protocols did not show a significant effect on viral replication. The present invention now demonstrates that a single heat treatment using an heated humidified air mixture of 43° C. for one and a half hours is sufficient to induce inhibition of viral replication in vivo. The treatment may of course be repeated after some time e.g. after one or more days or a week.

A humidified gas refers to a physiologically acceptable gas with a relative humidity (RH) of at least 70%, preferably at least 85%, more preferably at least 90%, such as 95, 96, 98, 99 or 100%. Preferred gases include air, oxygen or a combination of the two gases with or without added CO₂. In principle, viral replication in any type of tissue of a subject can be inhibited using the hyperthermic treatment according to the invention, provided that the tissue can be heated, such that the temperature of said tissue is raised to at least 2° C. above its normal physiological temperature. In a preferred embodiment, the tissue is a mucosal tissue heated via contact with a heated and humidified gas. Mucosal (or mucous) tissue lines some organs and body cavities (such as nose, mouth, lungs) and secretes mucus (a thick fluid) to prevent itself from becoming dry. Compared to other tissue types, mucosal tissues are more susceptible to become infected with infectious microbes, including viruses, because they lack the physical barrier of an intact skin. For example, many microbes gain access across the epithelia of the gastrointestinal, urogenital or respiratory tract. Because the heated gas is humidified the condensation of water vapour from the heated gas on the surface of the mucosal tissue aids the heat transfer to the tissue, furthermore, evaporation of moist (mucus) from the surface of mucosal tissue during the hyperthermic treatment, and therewith associated heat loss from the tissue, are both thought to be minimized in a method of the present invention. In a preferred embodiment, the heated gas is 100% saturated with water.

The temperature of the heated and humidified gas to be used in a method of the invention will of course depend on various factors, including the desired temperature of the tissue to be subjected to the hyperthermic treatment, the mode and route of administration of the gas and thermal sensitivity of the tissue(s) which are exposed to the gas. Thus, the temperature of the humidified gas to be delivered to the target tissue may be from 40° C.-50° C., preferably from 42° C.-48° C., more preferably from 42° C.-46° C., most preferably from 43° C.-44° C. Inhalation of warm air of 43° C.-44° C. and 100% saturated with water for 1.5 hours appears especially suitable for increasing the temperature of the tissue of the respiratory tract by at least 2° C. such that RNAi is enhanced, while at the same time minimizing the risk of preventing RNAi suppression and/or causing cell death induced by too harsh a temperature treatment (e.g. heating to above 42° C. for a substantial period of time, such as more than 45 minutes). However, other types of heat treatment according to the invention may require a higher or lower temperature of the introduced gas depending on the viral infection being treated and tissue in which the virus resides.

In one example, a method of the invention inhibits viral replication in a tissue located within the respiratory tract. The respiratory tract consists of an upper and a lower part. The upper respiratory tract (upper airway) consists of the nose, ears, mouth, sinuses, and throat. The lower respiratory tract consists of the trachea, the bronchial tubes, and the structures inside the lungs. Upon breathing, the air is drawn in through the nose or mouth and down through the trachea (windpipe). The trachea divides at its bottom end into two bronchi, one to each lung. Mucus in the bronchi serves to prevent the tissue from drying out and to trap and coat dust particles so they don't scratch or infect the delicate tissues in the lungs. The bronchi divide in the lungs into smaller branches called bronchioles. The tiniest bronchioles branch to the alveoli which are tiny, multi-lobed air sacs made of simple squamous cells. The thin wall of the alveoli enables air exchange with the equally-thin-walled capillaries of the circulatory system. In order to function properly, the alveoli must always stay moist.

Although most of the respiratory infections are in the upper airways, various types of microbial agents also injure the lung. In the upper airways, viral infections predominate. In a preferred embodiment, a method of the invention is used to combat a viral infection in the respiratory tract of a subject in need of such a treatment. To this end, infected tissue of the respiratory tract (including the trachea, the bronchi, the bronchioles and/or the alveoli) is contacted with a heated and humidified gas, such as air or oxygen, for a period of at least 1 hour.

In a method of the invention to inhibit viral replication in the respiratory tract, a heated, humidified gas is conveniently administered to a subject by inhalation, which may be through spontaneous or mechanical ventilation. Any kind of device or apparatus known in the art that is suitable to provide a person with hot moist gas (usually air or oxygen) at a controlled temperature can be used. These include invasive devices such as an endotracheal tube. Preferred is of course a non-invasive device. Of particular use in a method of the invention to inhibit viral replication in the respiratory tract is a respiratory rewarming apparatus typically employed for inhalation rewarming from hypothermia. See for example Morrison et al. 1979 J. Appl. Physiol. 1979, 46(6):1061 or the RES-Q-AIR non-invasive rewarming system from RESQ Products Inc. at Sooke, B. C., Canada at www.hypothermia-ca.com. Generally speaking, an inhalation rewarming apparatus comprises a water bath, a humidifier unit and heat exchanger, a temperature control and display unit, gas intake port and an outlet for the heated, humidified gas. The subject can inspire the gas through a mouth-and-nose-piece or a face mask. The flow rate can vary, depending on the subject and other conditions. In a preferred embodiment, the subject inhales heated and humidified gas at a flow rate of 10-80 l/min, preferably 15-50 l/min, more preferably 20-35 l/min.

In one embodiment, the subject inhales the heated and humidified gas with an increased ventilation rate. The ventilation rate of a subject at rest is typically around 10 l/min or even lower. At that rate the heating of the tissues especially the deeper lying (lung) tissue can be insufficient to reach the desired temperature. In a preferred embodiment the subject inhales the heated and humidified gas while hyperventilating. Hyperventilation is a state in which there is an increased amount of air entering the pulmonary alveoli. Inhalation of the heated and humidified gas in a method of the invention in a situation of hyperventilation maximizes respiratory heat exchange. Heat can penetrate into the deeper lying (lung) tissue. The subject may be asked to hyperventilate spontaneously however this may lead to dizziness. Alternatively, a situation of controlled (e.g. CO₂-assisted) hyperventilation in the subject can be created. For example, the apparatus may comprise a re-circulating loop such that the expired gas can be re-circulated through the heat exchanger. In a preferred embodiment of the invention, the subject first inhales heated and humidified gas without added CO₂ to heat the upper respiratory tract. After some time, for instance 10-30 minutes, CO₂ may be added to the gas (e.g. via re-circulation of expired air or from a gas cylinder) to assist hyperventilation in the subject and maximize the transfer of heat from the gas to the (preheated) tissue. The inspired CO₂ pressure (pCO₂) is preferably in the range of 5 to 7.5 kPa more preferably 6 to 7 kPa. The ventilation rate of the subject may be increased by exercising the subject. The use of exercise equipment like a home trainer bicycle or a roller band may help the subject to increase his ventilation rate.

In a preferred embodiment, a method of the invention comprises hyperthermic treatment of the respiratory tract using a heated gas which is 100% saturated with water or H₂O pressure (pH₂O) of at least 6,5 kPa, more preferably at least 7,5 kPa. This maximizes the heat capacity of the gas, delivers heat to the tissues through condensation and minimizes heat loss through evaporation. The water saturated gas may be in the form of a nebulized mist or a vapour. Mists are distinguished from gases containing water vapour by the presence of distinct liquid particles of small diameter, typically 1 to 5 microns. Preferably, a gas comprising water vapour is employed in the method of the invention. For a maximum effect of the inhaled heated gas, it is preferred that the subject minimizes the inhalation of cool (ambient) air following the heat inhalation procedure. Preferably, the heated and humidified air is inhaled before going to bed.

In one embodiment, herpes labialis (cold sores) may be treated using the method of the invention by heating the infected tissue (i.e. the lip/mouth region) of a subject in need of treatment to a temperature of at least 2° C. above the normal temperature of the said tissue for a period of at least 1 hour using a heated and humidified gas. This is easily achieved using an inhalation re-warming apparatus as described herein that is equipped with a face mask large enough to cover the infected area.

In a further aspect of the invention, there is provided use of an apparatus of the invention as described herein to supply a heated and humidified gas to a tissue or body part to inhibit viral replication in vivo. The apparatus is capable of producing humidified gas (e.g. air) at temperatures ranging from 37° C. to 50° C., and at a relative humidity of up to 100%, depending on the viral treatment being effected. More preferably, the apparatus provides a heated gas containing water vapour.

In a preferred embodiment, there is provided an apparatus for use in heating epithelial tissue of the respiratory tract of a subject suffering from a viral infection to a temperature of at least 2° C. above the normal physiological temperature of said tissue for a time period of at least 1 hour. Preferably an apparatus equipped with an air or gas re-circulation loop is employed so as to permit hyperventilation to occur and therewith maximize heat exchange between the heated gas and the epithelial tissues of the respiratory tract. Alternatively an apparatus of the invention is equipped with a simple CO₂ cylinder which in operation permits the controlled addition of CO₂ to the inhaled gas, such as air or O₂, thus causing the subject to hyperventilate in response to the increased level of CO₂ in the inhaled gas.

In a specific embodiment of the invention, there is provided a method for inhibiting the replication of herpesvirus in tissues of a subject mammal, such as a human being, that are infected with herpesvirus. According to the teaching of the invention, viral replication in the affected tissues may be inhibited by heating such tissues to at least 2° C. above their normal temperature (e.g. to 39-42° C.) for at least one hour. This is readily achieved by (locally) heating the affected tissues, for example, by use of heated pads or plasters placed on or in close proximity to the affected tissues. Preferably these pads or plasters contain a gel material that is readily formed in a shape that allows close contact with the virus affected area. Such gel pads or plasters comprising gels can be maintained at a suitable temperature (e.g. 40 to 43° C.) with a thermostatically regulated heater. This thermostatically regulated heater can be built into the said gel pads or plasters. Alternatively exothermic patches can be used such as ThermaCare Air-Activated Heat Wraps (available from Procter&Gamble).

In a further aspect of the invention, there is provided the use of heated elements, such as pads for heating virus-infected tissue of a subject to a temperature of at least 2° C. above the normal physiological temperature of said tissue for a period of at least 1 hour. Preferably an apparatus is used that is equipped with a thermostatically regulated heater which may be built into the pads.

It is preferred to start the antiviral heat treatment as provided herein as early as possible following initial signs and symptoms of virus infection. If viral replication is suppressed at an early stage following infection, the likelihood that the virus will be successfully eradicated by the body's immune system is increased. In the case of a respiratory infection an irritated nose or a scratchy throat is typically the first sign of infection, followed within hours by sneezing and a watery nasal discharge. Other symptoms of viral (e.g. herpes) infection include tingling, itching, burning or redness of the skin at the infection site.

It is further preferred to start the antiviral heat treatment as provided herein as early as possible following a known or suspected exposure to infection with a virus. If viral replication is suppressed at an early stage following infection, the likelihood that the virus will be successfully eradicated by the body's immune system is increased and the infection may go no further than the sub-clinical phase. Thus the treatment can be applied as a prophylactic treatment.

The concept of the present invention is based on the inhibition of viral replication through prolonged increased RNAi in the tissue, meaning that the capacity to silence genes via RNAi remains elevated for more than 6 and usually less than 96 hours (compared to the pre-treatment situation) after the heat treatment proper is stopped. Thus for the treatment of chronic viral infections the treatment is preferably repeated at regular intervals.

In a specific embodiment of the invention, there is provided a method for treating cervical dysplasia comprising applying heat to an area of the cervix using a heating apparatus of the invention as described herein. Cervical dysplasia is abnormal cell growth in the cervix. Cell growth is considered abnormal when some cervical cells appear immature compared with their normal-looking neighbours. Immature cervical cells also divide faster than expected, and their nuclei show specific types of microscopic change. Depending on the nomenclature used for different displasic conditions, cervical dysplasia may also be referred to as squamous intraepithelial lesion (SIL) or cervical intraepithelial neoplasia (CIN). Cervical dysplasia is an important health problem because it may progress to cervical cancer. Cervical dysplasia has also been linked to a very common virus called human papilloma virus (HPV). There are over 70 strains of HPV and more than one third of them can be sexually transmitted. Some strains cause warts, including genital warts, while others may lead to cancer. In particular HPV-16 and -18 are involved in cervical dysplasia and virus-infected cells may slowly progress to malignancy under the burden of episomal expression of HPV DNA (Melsheimer et al. Clin. Cancer Res. 2004, 10: 3059-3063). Cervical dysplasia can be diagnosed with regular Pap smears and when diagnosed at an early stage, cervical dysplasia can be treated to prevent it from developing into cancer. When a Pap smear is examined under a microscope and abnormal cervical cells involve the lower third of the specimen, the condition is described as “mild” cervical dysplasia (low-grade SIL or CIN1). If abnormal cervical cells are found on larger portions of the specimen, the condition is described as “moderate” or “severe” cervical dysplasia (high-grade SIL or CIN 2 or 3). Treatment for cervical dysplasia varies from one woman to another, depending on the location and size of the lesion, and whether it's low grade or high grade. Known therapies include destruction of the lesion by cryo-therapy or Laser treatment and removal of the lesion by a loop electrosurgical excision procedure (LEEP) or by a cone biopsy, which removes a cone-shaped piece of tissue from the opening of the cervix. The present invention now provides a method for inhibiting the replication of HPV in cervical cells by heating the affected area to at least 2° C. above its normal temperature (e.g. to 39-42° C.) for at least one hour. This is readily achieved by locally heating the affected area, for example, by use of a heated thermostatically regulated probe or by other methods known in the art. By treating the affected area of the cervix daily, or every other day or twice weekly for two, three or more weeks, the burden of HPV infection is significantly reduced such that the progress to malignancy is stopped or even reversed. It is of particular importance to treat the cervix in the pre-malignant stages, preferably low grade SIL or CIN1. In these stages the cell growth usually still depends on the presence of the virus, while in the malignant stages the tumour cell growth may become virus independent. The heat treatment according to the invention may be combined with known therapies for cervical dysplasia. Regional hypothermia is known in the art to treat cancers, including cervical cancer (de Wit et al. Br. J. Cancer, 1999, 80(9): 1387-1391). However, this anti-cancer treatment is based on the observation that tumour cells are more sensitive to heat-induced apoptosis, particularly in combination with cytotoxic agents or radiation. The objective of the anti-cancer hyperthermic treatment is to apply sufficiently high temperatures (e.g. over 42° C.) to promote cell death of tumour cells. In contrast, the present invention involves a heat treatment of non-tumour, pre-malignant cells to enhance the RNAi mechanism in the cells which inhibits virus replication and so potentially if not actually avoid reaching the malignant stage. Furthermore, a temperature of over 42° C. is typically required for the induction of (tumour) cell death whereas preferred temperatures for inhibiting viral replication are 39-42° C., more preferably 39-40° C. Naturally, the skilled addressee will appreciate that too high a temperature that may induce cell death should be avoided in the application of the present invention.

The concept of the present invention is based on the inhibition of viral replication through enhancing RNAi, an antiviral defence mechanism of the infected host cell which leads to silencing of viral genes. However, the application of the in vivo heat treatment according to the invention is not necessarily limited to enhancing antiviral gene silencing. It is also advantageously used if RNAi-mediated silencing of an endogenous or an exogenous gene of interest is desired upon introduction of dsRNA homologous to the gene into the host cell. Because an RNAi sequence seeks out and destroys its target without affecting other genes, the RNAi effect can be highly selective. It is widely speculated in the medical community that clinical applications of RNAi are potentially endless: any gene whose expression contributes to disease (disease gene) is a potential target, from viral genes to oncogenes to genes responsible for heart disease, Alzheimer's disease, diabetes, and many more. In order to enhance the RNAi effect of a dsRNA which is introduced into a cell in situ (either by injection, genetic engineering techniques, and/or using a viral or non-viral delivery vehicle) heat treatment according to the invention may be contemplated.

Herewith, the invention provides a method to enhance RNAi-mediated gene silencing in vivo in a tissue, comprising stably increasing RNAi in said tissue by providing heat to said tissue. In a preferred embodiment, tissue is heated to a temperature of at least 2° C. above its normal physiological temperature for a duration of 1-4 hours, preferably 1-2 hours, more preferably 1-1.5 hours. A method of the invention is advantageously used to enhance in vivo silencing of a disease gene.

According to the present invention there is also provided apparatus for inhibiting the replication of a virus in the tissue of the respiratory tract of a subject, comprising:

a gas inlet for receiving a gas in said apparatus,

a heating device for heating said gas,

a humidifying device for humidifying said gas,

gas guiding means for guiding said gas from the gas inlet to the heating device and to the humidifying device and then to a gas outlet for releasing said heated and humidified gas,

a ventilation rate increasing device for increasing the ventilation rate of the subject, and

a temperature controller which is connected to said heating device and controls the temperature of the tissue of the respiratory tract of the subject so that in use at least part of said tissue is heated to a temperature of at least 2 and no more than 9° C. above its normal physiological temperature.

As discussed above the ventilation increasing device may comprise CO₂ adding means for introducing CO₂ into said gas before it is released from said gas outlet or exercise equipment to be used by the subject. The apparatus may further comprise an oxygen adding device for introducing oxygen into said gas before it is released from the gas outlet. The gas outlet may be provided with a mouth-and-nose piece or face mask.

According to the invention there is further provided apparatus for inhibiting the replication of a virus in the tissue of the rectal tract, the female reproductive tract, cervix or vagina of a subject comprising:

a penetrating element for penetrating the rectal tract, the female reproductive tract, cervix, or vagina of the subject which comprises an outer wall,

a second heating device in use to heat the tissue surrounding said penetrating element,

a second temperature controller which is connected to said second heating device and controls the temperature of the surrounding tissue so that in use at least part of said tissue is heated to a temperature of at least 2 and up to 9° C. above its normal physiological temperature.

The present invention may also be used to treat body extremities so according to a yet further aspect of the present invention there is also provided apparatus for inhibiting the replication of a virus in the tissue of an extremity of a subject the apparatus comprising:

a receiving element for receiving the extremity of the subject, which receiving element comprises an inner wall and in use at least partly surrounds the extremity of the subject,

a third heating device for heating the tissue which is surrounded by the receiving element, and

a third temperature controller which is connected to said third heating device and controls the temperature of said tissue of the extremity of a subject so that in use at least part of said tissue is heated to a temperature of at least 2 and up to 9° C. above its normal physiological temperature.

In the different apparatuses discussed above the second and third heating devices may be similar in form and function, and what is described in respect of one may apply to the other. Similarly other parts such as the second and third temperature controllers may also be equivalent. The heating devices may comprise liquid heating means for heating a liquid, and liquid guiding means for guiding said liquid between said liquid heating means and the penetrating or receiving element. The penetrating and receiving elements may each include a duct through which warmed liquid is pumped by pumping means to heat the outer wall or inner wall respectively.

The outer wall of the penetrating element and the inner wall of the receiving element may comprise an expandable or flexible material, which preferably is elastic. Such an expandable or flexible material may be expanded such that a substantially complete contact with the surface of tissue to be treated with the method of the invention is achieved. Naturally the skilled addressee will appreciate that the expansion of the said flexible material may be effected by the regulation of the flow of liquid and/or gas by means of a pump.

The outer and inner walls may also comprise protrusions that extent toward the tissue. The penetrating element and the receiving element may be provided with a vibrating device for vibrating them, as this improves thermal contact with the tissue.

The temperature controllers of each embodiment of apparatus may further include time controllers for controlling the time period during which the tissue is heated for an effective time interval depending on the treatment being effected.

Certain aspects of the invention will now be described in detail with reference to the accompanying drawings in which:

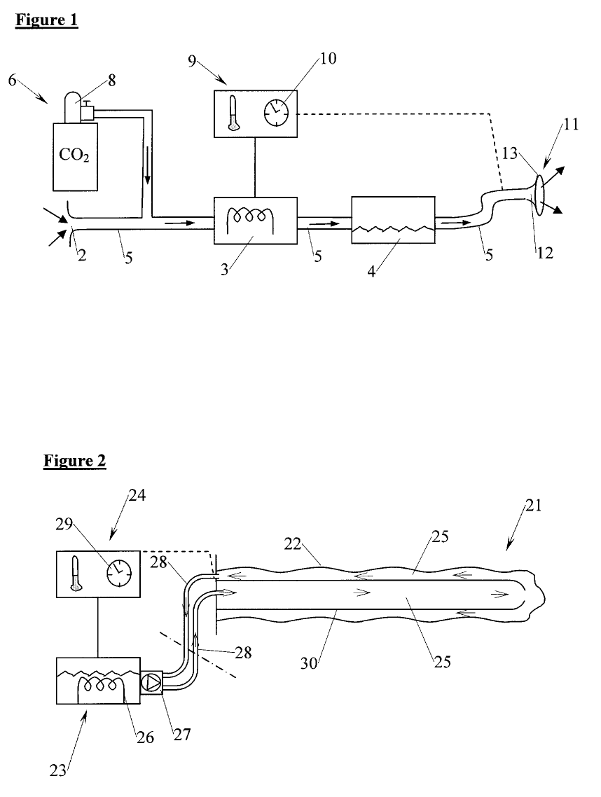

FIG. 1 shows a schematic view of an embodiment of the apparatus for inhibiting the replication of a virus in the tissue of the respiratory tract of a subject,

FIG. 2 shows a schematic view, partly in cross-section, of a second embodiment of the apparatus for inhibiting the replication of a virus in the tissue of the rectal tract or the female reproductive tract, such as the cervix or vagina of a subject,

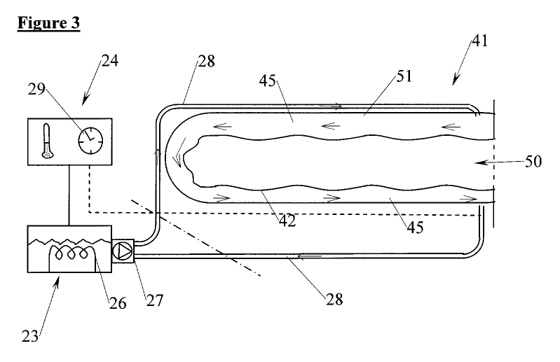

FIG. 3 shows a schematic view, partly in cross-section, of a third embodiment of the apparatus for inhibiting the replication of a virus in the tissue of the extremity of a subject.

FIG. 1 shows an embodiment of the apparatus for inhibiting the replication of a virus in the tissue of the respiratory tract of a subject, which apparatus comprises a gas inlet 2 for receiving a gas. In the embodiment of FIG. 1 air is received by the gas inlet 2 from the ambient, but it is also possible to connect the gas inlet 2 to a gas container which contains a specific gas (preferably oxygen) or a mixture of specific gases. The airflow into the gas inlet 2 is shown by the accompanying arrows, as is the subsequent flow of the air through the apparatus. The received air is guided by gas guiding means 5 to the heating device 3 which heats the air. The heating device 3 is connected by the gas guiding means 5 to the humidifying device 4, which humidifies the air. Between the gas inlet 2 and the heating device 3 the gas guiding means 5 are connected to a ventilation rate increasing device 6 which comprises a CO₂ gas unit 8. The CO₂ gas unit 8 can be used to add CO₂ to the air passed to the subject so that in use the ventilation rate of the subject will increase. Instead of altering the gas component to increase the ventilation rate, the ventilation rate increasing device 6 can comprises exercise equipment for the subject, use of which causes the breathing rate to increase.

The heating device 3 heats the gas before it passes to the humidifier to ensure suitable humidity can be achieved in the air passing therethrough. The air passing into the humidifier may be at the temperature required for delivery to the tissue, or may be at a higher temperature. The gas guiding means 5 for the air leaving the heating device and/or the humidifying device may be adapted to maintain the gas at the required temperature or may allow it dissipate heat to reach the desired temperature. The gas guiding means extend to the gas outlet 11. The airflow out of the gas outlet 11 is shown by the arrows.

The apparatus further comprises a temperature controller 9, which is connected to the heating device 3 for controlling the heating device 3. The temperature controller 9 comprises a time controller 10 for controlling the time period during which the temperature controller 9 activates the heating device 3. The temperature controller may also be connected to the last part of the gas guiding means 5 before the outlet 11, so that it may monitor the temperature of the gas before it passes to the subject. This can be particularly important if the gas is supplied to the humidifying device 4 at a higher temperature than that at which it is to be provided to the subject, to make sure it has been allowed to cool sufficiently.

The gas outlet 11 includes a face mask 13 with an elastic retaining band 12 so that the face mask 13 can be held in place on the face of the subject. In use the subject will breathe in the heated and humidified mixture of air and CO₂ so that the temperature of the tissue of the respiratory tract will rise to a specific temperature and for a specific time period. In this way the replication of a virus in the tissue of the respiratory tract of the subject will be inhibited.

FIG. 2 shows a second embodiment of the apparatus for inhibiting viral replication in the tissue of the rectal tract or the female reproductive tract, including the cervix or vagina, of a subject. The apparatus comprises a penetrating element 21, which in use penetrates the rectal tract or the female reproductive tract of the subject. The penetrating element 21 comprises an outer wall 22 which is flexible and elastic. Inside the outer wall 22 of the penetrating element 21 there is a hollow core 30 with a duct 25 formed therein. In use liquid flows in through this duct 25, and then out through the space between the hollow core 30 and the outer wall 22. The flexible outer wall 22 allows the the form of the penetrating element 21 to adjust in use to the form of the surrounding tissue. The elastic nature of the outer wall allows it to be inflated, so that the size thereof can also be adjusted. The outer wall can also have protrusions (not shown) which can provide a larger contact surface between the penetrating element 21 and the surrounding tissue without lengthening the penetrating element 21.

The embodiment shown in FIG. 2 further comprises a second heating device 23. The second heating device 23 comprises liquid heating means 26 and pumping means 27 to drive heated liquid through the liquid guiding means 28 and into the penetrating element 21. The second heating device 23 is connected to the penetrating element 21 by the liquid guiding means 28 so that heated liquid may be passed to and from the penetrating element 21. Part of the diagram including the whole penetrating element 21 is shown in a cross-sectional view. The arrows in the diagram show the flow of liquid within the apparatus. The liquid guiding means 28 transfer the heated liquid from the second heating device 23 to the duct 25. The heated liquid then passes through the penetrating element 21 and returns to the second heating device 23 through the return part of liquid guiding means 28. Inside the duct 25 liquid flow controlling means (not shown) are arranged to ensure a full circulation of the liquid through the duct 25.

The apparatus of the second embodiment also comprises a second temperature controller 24, which is connected to and controls the second heating device 23. The second temperature controller 24 includes a second time controller 29 for controlling the time period in which the second temperature controller 24 activates the second heating device 23.

In use the penetrating element 21 element is place in the rectal tract or the female reproductive tract of the subject. The flow of the heated liquid through the penetrating element 21 causes the outer wall 22 to be heated to the desired temperature. In this way the penetrating element 21 heats the nearby surrounding tissue for a specific time period so that viral replication in said tissue is inhibited.

The apparatus can also comprise a vibrating device for vibrating the penetrating element 21. In this way a better thermal contact between the penetrating element 21 and the surrounding tissue is achieved. FIG. 3 shows a third embodiment of the apparatus for inhibiting viral replication in the tissue of a body extremity of a subject. The apparatus comprises a receiving element 41 for receiving an extremity of the subject. The body part extremity may be a hand, foot, scrotum and/or a phallus. In use the body part extremity is inserted in the receiving element 41 which at least partly surrounds the extremity. The receiving element 41 comprises an outer sleeve 51, defining an opening 50 which gives access to the inside thereof. An inner wall 42 which is flexible and elastic, is located within the outer sleeve 51 in such a way that a second duct 45 is formed there between, through which liquid can flow. Inside the second duct 45 liquid flow controlling means (not shown) are arranged to ensure a full circulation of the liquid through the receiving element 41 and hence around the extremity.

The flexible inner wall 42 will in use adjust to the form or contour of the received body extremity, and as it is elastic it can be inflated, so that the size thereof can be adjusted to accommodate smaller extremities. The inner wall may comprise inwardly-directed protrusions which enable a better grip or a better contact between the receiving element 41 and the tissue of the received body extremity.

The apparatus of this third embodiment further comprises a mechanism for supplying heated liquid that is the same as that described with respect to FIG. 2. Like reference numerals have been used to describe like parts. The heating device 23 pumps the heated liquid through liquid guiding means 28 to and from the receiving element 41. The arrows in the part of the diagram that is shown in cross-section show the flow of liquid.

In use the extremity of the subject is placed into the receiving element 41 through the opening 50. The heating device pumps heated liquid through the second duct 45 in the receiving element 41, which causes the inner wall 42 thereof to heat up. In this way the receiving element 41 heats the tissue of the surrounded extremity for a specific time period so that viral replication in said tissue is inhibited. The apparatus can also comprise a vibrating device for vibrating the receiving element 41 as this improves thermal contact between the receiving element 41 and the surrounded tissue.

The liquid used in the second and third embodiments may be any suitable type, with appropriate viscosity, thermal capacity and toxicity. Water or light mineral oil are suitable.

The thermal contact may be further improved by providing a heat conducting medium between the tissue and the contacting wall (eg the outer wall of the penetrating element or the inner wall of the receiving element) of the apparatus. Improved thermal contact between the penetrating element or receiving element and the adjacent tissue improves the efficiency of the present invention. This heat conducting medium can also improve the comfort of use, and may be gel, saliva, water, lubricant etc.

1.-29. (canceled)

30. A method for treating a viral disease in a mammal in need of such treatment, which comprises heating cells of virus-infected tissue of the said mammal with a heating apparatus to a heating temperature of at least 2° C. higher than that of the normal physiological body temperature for the said tissue.

31. A method according to claim 30 wherein the said heating temperature lies in the range from 2° C. to 13° C. above that of the normal physiological body temperature for the said tissue

32. A method according to claim 30 wherein the virus-infected tissue is selected from epithelial cells of the lungs, nasal passages, trachea, bronchioles, alveoli, and body parts such as the feet, hands, skin, sexual organs and parts thereof such as the penis and scrotal sac, vulva, uterus, cervix, clitoris, vulva (labia major and/or labia minor), perineum and surrounding infected skin.

33. A method according to claim 30 wherein the mammal is a human being.

34. A method for inhibiting the replication of a virus in virus-infected cells of a tissue of a subject mammal in vivo that comprises heating said virus-infected tissue to a temperature in the range of from at least 2° C. to 9° C. above the normal physiological temperature of said tissue for at least 1 hour.

35. A method according to claim 34 wherein the mammal is a human being.

36. A method for inhibiting the replication of viruses in vivo in virus-infected tissue of a subject mammal, comprising heating said tissue to a temperature of at least 6° C. but not more than 9° C. above its normal physiological temperature for up to 45 minutes such that apoptosis in the tissue is not induced.

37. A method according to claim 36 wherein the mammal is a human being.

38. A method according to claim 37 wherein the temperature of heating lies in the range of from 39° C. to 42° C. and is applied over a time interval of at least 60 minutes.

39. A method according to claim 37 wherein the temperature of heating lies in the range of from 43° C. to 46° C. and is applied over a time interval of less than 60 minutes.

40. A method according to claim 30 wherein the heat to the cells is supplied in the form of a heated humidified gas having a relative humidity of at least 70%, optionally comprising CO₂, or in the form of a heated element.

41. Apparatus for inhibiting the replication of a virus in the tissue of the rectal tract, the female reproductive tract, cervix or vagina of a subject comprising
a penetrating element (21) for penetrating the rectal tract, the female reproductive tract, cervix or vagina of the subject, which penetrating element (21) comprises an outer wall (22),
a second heating device (23) in use to heat the tissue surrounding said penetrating element (21), and
a second temperature controller (24) which is connected to said second heating device (23) and controls the temperature of the surrounding tissue so that in use at least part of said tissue is heated to a temperature of at least 2 and up to 13° C. above its normal physiological temperature.

42. Apparatus according to claim 41 in which said second heating device (23) comprises liquid heating means (26) for heating a liquid, and liquid guiding means (28) for guiding said liquid between said liquid heating means (26) and the penetrating element (21), said penetrating element (21) comprising a duct (25) through which said liquid is pumped by pumping means (27) to heat the outer wall (22).

43. Apparatus according to claim 41, in which said outer wall (22) comprises an expandable and/or flexible material, which preferably is elastic.

44. Apparatus according to claim 41, in which said outer wall (22) comprises protrusions.

45. Apparatus according to claim 41, in which said apparatus comprises a vibrating device for vibrating said penetrating element (21).

46. Apparatus according to claim 41, in which said second temperature controller (24) includes a second time controller (29) for controlling the time period during which said tissue is heated.

47. Apparatus for inhibiting the replication of a virus in the tissue of an extremity of a subject comprising:
a receiving element (41) for receiving the extremity of the subject, which receiving element (41) comprises an inner wall (42) and in use at least partly surrounds the extremity of the subject,
a third heating device (43) for heating the tissue which is surrounded by the receiving element (41), and
a third temperature controller (44) which is connected to said third heating device (43) and controls the temperature of said tissue of the extremity of a subject so that in use at least part of said tissue is heated to a temperature of at least 2 and up to 13° C. above its normal physiological temperature.

48. Apparatus according to claim 47, in which said third heating device (43) comprises second liquid heating means (46) for heating a liquid, and second liquid guiding means (48) for guiding said liquid between said third heating device (43) and penetrating element (41), said penetrating element comprises a second duct (45) through which in use said liquid is pumped by second pumping means (47) for the heating of said inner wall (42).

49. Apparatus according to claim 47, in which said inner wall (42) comprises an expandable and/or flexible material, which preferably is elastic.

50. Apparatus according to claim 47, in which said inner wall (42) comprises second protrusions.

51. Apparatus according to claim 47, in which the apparatus comprises a second vibrating device for vibrating the receiving element (41).

52. Apparatus according to claim 47, in which said third temperature controller (44) comprises a third time controller (49) for controlling the time period during which said tissue is heated.

53. Apparatus for inhibiting the replication of a virus in the tissue of the respiratory tract of a subject, comprising:
a gas inlet (2) for receiving a gas in said apparatus,
a heating device (3) for heating said gas,
a humidifying device (4) for humidifying said gas,
gas guiding means (5) for guiding said gas from the gas inlet (2) to the heating device (3) and to the humidifying device (4) and then to a gas outlet (11) for releasing said heated and humidified gas,
a ventilation rate increasing device (6) comprising CO₂ adding means (8) for introducing CO₂ into said gas before it is released from said gas outlet (11) for increasing the ventilation rate of the subject and
a temperature controller (9) which is connected to said heating device (3) and controls the temperature of the tissue of the respiratory tract of the subject so that in use at least part of said tissue is heated to a temperature of at least 2 and no more than 13° C. above its normal physiological temperature.

54. Apparatus according to claim 53, in which the apparatus further comprises an oxygen adding device for introducing oxygen into said gas before it is released from the gas outlet (11).

55. Apparatus according to claim 53, in which the gas outlet (11) comprises a mouth-and-nose piece or a face mask (13).

56. Apparatus according to claim 53, in which said temperature controller (9) comprises a time controller (10) for controlling the time period for which said tissue is heated.

57. Apparatus for inhibiting the replication of a virus in the tissue of the respiratory tract of a subject, comprising:
a gas inlet (2) for receiving a gas in said apparatus,
a heating device (3) for heating said gas,
a humidifying device (4) for humidifying said gas,
gas guiding means (5) for guiding said gas from the gas inlet (2) to the heating device (3) and to the humidifying device (4) and then to a gas outlet (11) for releasing said heated and humidified gas,
a ventilation rate increasing device (6) comprises exercise equipment to be used by the subject,
a temperature controller (9) which is connected to said heating device (3) and controls the temperature of the tissue of the respiratory tract of the subject so that in use at least part of said tissue is heated to a temperature of at least 2 and no more than 13° C. above its normal physiological temperature.

58. Apparatus according to claim 57, in which the apparatus further comprises an oxygen adding device for introducing oxygen into said gas before it is released from the gas outlet (11).

59. Apparatus according to claim 57, in which the gas outlet (11) comprises a mouth-and-nose piece or a face mask (13).

60. Apparatus according to claim 57, in which said temperature controller (9) comprises a time controller (10) for controlling the time period for which said tissue is heated.
